(19) United States
(12) Patent Application Publication
(10) Pub. No.: US 2003/0040095 A1
(43) Pub. Date: Feb. 27, 2003

(54) Method for the production of pharmaceutically active recombinant proteins

(76) Inventors: Achille Arini, Morcote (CH); Raffaella Coppolecchia, Morcote (CH); Francesca Paola Pagani, Milan (IT); Detlev Herbst, Lugano (CH); Antonio Tognini, Locarno (CH)

Correspondence Address: James V. Costigan, Esq., HEDMAN & COSTIGAN, P.C., Suite 2003, 1185 Avenue of the Americas, New York, NY, 10036-2646, US

(21) Appl. No.: 09/815,533

(22) Filed: Mar. 16, 2001

(51) Int. Cl. C12P 21/02; C12N 9/72; C12N 5/06

(52) U.S. Cl. 435/215; 435/69.1; 435/320.1; 435/358

(57) ABSTRACT

The present invention discloses a method for the production of a mature recombinant protein into the culture medium of an eukaryotic cell line genetically transfected with a cloned precursor cDNA sequence, which comprises an incubation of said cell line in the cell culture medium wherein alkanoic acids, their derivatives or salts thereof have been added for a period of time of at least 24 hours. The process allows the secretion of recombinant proteins into the culture medium in their physiologically active (mature) form.

### FIELD OF THE INVENTION

The technical field of the invention is the production of recombinant proteins by means of genetic engineering of eukaryotic cells.

### BACKGROUND ART

Many enzymes or hormones are produced by the cell as inactive pro-enzymes or pro-hormones and are subsequently converted in vivo at the site of “use” or at the “moment of use” into the corresponding active substances. Examples of such enzymes are some matrix metallo proteases, such as collagenase I, plasminogen, chymotrypsinogen, trypsinogen. This mechanism of “activation” is also common to hormones such as chromogranin, calcitonin etc.

Among the molecules synthesized by the cell as precursors, Urokinase (urinary type Plasminogen Activator, u-PA) can be considered a typical example. This enzyme is a plasma glycoprotein belonging to the wide family of serine proteases. Its main function, among many different ones, is the physiological activation of plasminogen to plasmin (Barlow, G. H, Methods in Enzymology, 45: 239-244, (1976)). Plasmin is a key proteolytic enzyme in the fibrinolytic process that leads to lysis of a clot (Robbins, K C and Summaria, L, Methods in Enzymology, 45: 257-273, (1976)).

In humans u-PA is expressed in different tissues as pre-proenzyme (Pre-prourokinase) and is then secreted in the blood as Prourokinase in a single-chain form. This zymogen form is therefore frequently abbreviated with the term sc-uPA (single chain u-PA). sc-uPA has an apparent molecular weight on SDS-PAGE of 50000-54000 Dalton and is catalytically inactive.

Within the blood plasmin performs a proteolytical cleavage which converts the proenzyme sc-uPA into a two-chain polypeptide, named tc-uPA, which is catalytically and physiologically active.

tc-uPA is composed of an A- and a B-chain, linked to each other by a disulfide bond. The B-chain harbors the catalytical site, as well as the N-glycosylation site, located at Asn³⁰² (Bergwerff A A, van Oostrum J, Kamerling J P and Vliegenthart J F, Eur. J. Biochemistry,1995, 228: 1009-1019). This form of uPA is called two chain High Molecular Weight uPA (tc-uPA HMW). A further proteolytical cleavage operated by plasmin on tc-uPA HMW A-chain, leads to the formation of a shorter two chain Urokinase molecule called tc-uPA LMW (two chain u-PA Low Molecular Weight).

Various attempts in the production of tc-uPA by the recombinant DNA technologies confirm on one side the relevance of such a molecule in the clinic and on the other side the need of such an approach required mainly for safety and purity reasons.

EP 154272 describes the production of recombinant glycosylated scuPA obtained by insertion of the cDNA sequence in animal cells.

EP 303028 describes the production of recombinant glycosylated scuPA obtained by insertion of the genomic sequence into mammalian cells.

The production described in these patents refers to recombinant Urokinase (sc-uPA) in the enzymatically inactive form.

Production of the active enzyme (tc-uPA) by recombinant DNA is still an open question, mainly because of its complex extracellular processing. As a matter of fact, low amounts of recombinant active tc-uPA is obtained in some of the recombinant eukaryotic systems described so far. In these eukaryotic systems the resulting product is a mixture of sc-uPA and tc-uPA (Cheng D et al., Chinese Journal of Biotechnology, 1994, 9: 151-159). This is mainly due to the inefficiency of the processing steps involved in sc-uPA activation This fact raises some problems for the purification of the two individual forms.

The clinical use of a mixture of HMW and LMW uPA would otherwise involve the problems of a precise reproducibility of the ratio of the relative components, which, moreover, exhibit different pharmacological properties.

On the other side, the production of the recombinant sc-uPA led to a different therapeutical strategy, involving the administration of sc-uPA as such subsequently converted into the active enzyme within the blood stream, by the endogenous plasmin. In this case the problems arising are due to the uncertainties of the dosage, because of the unpredictability of the conversion rate in vivo.

At present, however, the molecular HMW and LMW tc-UPA forms remain the only molecules of proven therapeutic interest, based on their successful use for many years in the clinical treatment of the clot lysis.

Both molecules, that are currently available as pharmaceutical products, are not recombinant (for a review see: Scripp's thrombolytic report, PJB Publications Ltd., (1993)). The HMW form is currently produced by extraction from human urine, as described for example in DE3439980, whereas the LMW form has been produced from human foetal kidney cell cultures in the presence of serum as described in DT2551017. In this case serum does not only supply growth factors to the cells, but also plasmin for the conversion of Prourokinase into the tc-uPA LMW form. However, safety concerns have been recently raised toward this product, due to its derivation from primary cells (“The pink sheet”, Feb. 1, 1999, page 6).

### SUMMARY OF THE INVENTION

The present invention is related to a method for the production of mature recombinant proteins by genetically transfected eukaryotic cell lines. The maturation process of the recombinant precursor is achieved by growing the cell line in the presence of alkanoic acids or their derivatives or salts thereof for at least 24 hours. According to a preferred embodiment of the invention, the mature recombinant protein is the two chain-uPA (tc-uPA), expressed from a cloned cDNA precursor.

Further object of the invention is therefore a process for the production isolation and purification of recombinant HMW and LMW tc-uPA and the products derived, corresponding to the two forms of mature Urokinase, obtained accordingly to such processes.

Further objects of the present invention are represented by a method for the treatment of thromboembolytic disorders, which makes use of the recombinant mature HMW and LMW tc-uPA and pharmaceutical compositions comprising as active agents the recombinant HMW and LMW tc-uPA obtained according to the process of the invention.

### DETAILED DESCRIPTION OF THE INVENTION

The present invention is related to a method for the production of mature recombinant proteins into the culture medium of genetically modified eukaryotic cells.

The Applicant has now surprisingly found that when genetically modified eukaryotic cells are treated for a time of at least 24 hours with alkanoic acids or their derivatives or salts thereof which are added to the cell culture media, the conversion of a recombinant precursor protein into the corresponding mature form is very efficient and a mature active protein is accumulated at high amount into the cell culture supernatant.

The conversion of a precursor protein into its mature form is particularly efficient when the treatment with alkanoic acids or their derivatives or salts thereof, is combined with the lowering of the cell culture temperature to values equal or below 37° C., preferably comprised between 30° C. and 36° C., most preferably 34° C., indicating a synergetic effect.

The term genetically manipulated cells refers to cells which have been transfected or transformed with exogenous DNA, preferably cDNA, encoding for a desired precursor protein. In a preferred embodiment the cDNA sequence is the nucleic acid encoding for the Urokinase precursor (Pre-prourokinase or Prourokinase). The term precursor protein refers to a polypeptide which is secreted or otherwise produced by the cell and which needs further proteolytic cleavage or “processing” for its activation, or for its production in a form as close as possible to the biologically active or natural form. Other examples of such precursor proteins encoded by the corresponding DNA or cDNA sequence are zymogens such as: trypsinogen, chymotrypsinogen, plasminogen, proamylase, prolipase, matrix metallo proteases, i.e. collagenase I, factors belonging to the cascade of the complement system, and prohormones such as: pro-hepatocyte growth factor (pro-HGF), pre-proinsulin, somatostatin, chromogranin-A etc. In a preferred embodiment the precursor protein is a pre-pro-enzyme and the most preferred is Pre-prourokinase or Prourokinase, which becomes active in the form of tc-uPA (two chain Urokinase). Urokinase (uPA) is a serine protease whose main function in vivo is the activation of plasminogen to plasmin. In humans uPA is expressed in different tissues as pre-proenzyme and then secreted in the blood as Prourokinase, catalytically inactive, in a single chain form (sc-uPA), which is in turn processed by plasmin into a two chain uPA (tc-uPA), catalytically active. The tc-uPA is composed by a A- and a B-chain, the latter carrying the catalytic site, linked to each other by a disulphide bond. Active tc-uPA is naturally found in the blood and urine in two different forms: the tc-HMW and LMW uPA, which differ in their A-chain, shorter in tc-LMW uPA.

Alkanoic acids, or their derivatives or salts have until now been used in the prior art to enhance the yield of production/secretion of recombinant proteins in mammalian culture systems. According to the present invention, alkanoic acids, or their derivatives or salts thereof act as “processing enhancers”. To the best of our knowledge this is the first time that alkanoic acids or their derivatives or salts thereof are used as “processing enhancers” of precursor proteins as defined above.

Alkanoic acids or their salts or derivatives thereof are preferably a C₂-C₁₀, and more preferably a C₃-C₄ straight chain. Alkanoic acids or their salts and/or derivatives are preferably chosen among butyrate or propionate, preferably in their sodium-salts, or tributyrin or phenyl-butyrate. Especially preferred is sodium butyrate.

Alkanoic acids or their derivatives are added to the culture medium of genetically modified cells in concentration comprised between 0.01 and 500 mM. In the case of mammalian cells the preferred concentration range is comprised between 0.1 and 20 mM, most preferred is 0.5-2.5 mM. It will be appreciated however, that such concentrations may be varied according to the cell line used and according to other factors such as the viability of the cell culture during or at the end of treatment. According to a preferred embodiment, eukaryotic cells are mammalian cells chosen among those commonly used for the production of recombinant proteins: HEK-293 cells, CV-1, COS, BSC-1, MDCK, A-431, BHK, CHO. In a preferred embodiment CHO cells are CHO-Messi cells (ECACC N° 93080520).

A further object of the present invention is a process for the production of recombinant human tc-uPA in CHO cells, comprising the addition of alkanoic acids or their derivatives or salts thereof to a serum-free culture medium where the CHO cell line, genetically modified with an eukaryotic expression vector carrying the Pre-prourokinase cDNA, is maintained for a time of at least 24 hours at a temperature equal or lower than 37° C., preferably comprised between 33 and 35° C., most preferably 34° C. Further object of the present invention is a process for the conversion of Prourokinase (sc-uPA) to tc-urokinase (tc-uPA) by addition of alkanoic acids, or their derivatives, or salts thereof into the culture media of CHO cells and fermentation of the cell culture at temperatures equal or lower than 37° C. and for a period of time of at least 24 hours.

As said above for the natural form, also for recombinant Urokinase the term tc-uPA refers to the catalytically active Urokinase, which can be in the form of HMW and LMW tc-uPA. The HMW and LMW tc-uPA differ for a differentially processed A-chain and show very similar functional activities. The HMW and LMW tc-uPA may be distinguished e.g. for analytical purposes for a different electrophoretic pattern onto a not-reducing SDS-PAGE: HMW tc-uPA migrates at 50-54 KD, while LMW tc-uPA migrates at 30-33 KD under the same conditions.

Further object of the present invention is a process for the production of recombinant human tc-uPA which is obtained through a process comprising the following steps:

a) culturing genetically manipulated CHO cells transfected with the pre-proUK cDNA or gene in a culture media comprising alkanoic acids or their derivatives or salts thereof, preferably butyrate or its salts in concentration comprised between 0.1 and 20 mM and at a temperature comprised between 30° C. and 37° C., even more preferred comprised between 33° C. and 35° C., most preferred 34° C.;

b) continuing said culture for a period of time of at least 24 hours, or preferably comprised between 48 and 200 hours, most preferably about 120 hours (about five days) and

c) recovering the cell culture supernatant for the isolation of said recombinant human tc-uPA.

According to a preferred embodiment of the process of the invention, the genetically manipulated cell line is a stable CHO transformant, most preferably a CHO-Messi (ECACC N. 93080520) transformant selected on the basis of the acquisition of a metabolic marker gene. The culture media is preferably a serum-free culture medium, even more preferably a serum- and protein-free defined medium, such as those commercially available. In a preferred embodiment the cell-culture media is CHOMaster®.

For stable CHO transformant or transfectant is intended a CHO clone transfected with an expression vector which is stably integrated into the cell genome. The eukaryotic expression vector is chosen accordingly to criteria well known in the art: the presence of a strong eukaryotic or viral promoter, such as CMV-IE, SV40 late or early promoter, RSV to drive the transcription of the exogenous DNA; a polyadenylation signal; enhancers of transcription and other regulatory regions which are chosen according to methods well known in the art. Other features of the expression vectors are: a prokaryotic origin of replication, a gene for selection in eukaryotic as well as in prokaryotic cells, such as the β-lactamase gene or kanR, or neoR, or tet-F or Hygromycin-R, as well known by the skilled artisan.

According to a preferred embodiment of the invention, selection in eukaryotic cells is performed by expression of the metabolic marker gene: Trp-Synthase (trpB gene) on Trp-auxotrophic CHO cells, or by Histidinol dehydrogenase (hisD gene) on His-auxotrophic CHO cells.

Stable clones are preferably selected on the basis of their growth properties, productivity levels and their stability in culture.

Cultivation of the CHO selected clone is usually performed in bioreactor according to protocols well known in the art. According to a preferred embodiment the cultivation is performed in batch. The preferred initial cell concentration is about 3×10⁵ living cells/ml and the cell viability of the inoculum is usually higher than 95%, as measured by the Trypan-Blue exclusion dye method. Usually, the ratio “volume of inoculum/volume fresh medium” varies between 1:1 and 1:5, according also to the total capacity of the bioreactor and to the cell growth.

At a cellular density comprised between 1×10⁶ and 4×10⁶ cells/ml, usually occurring after 3-5 days of growth, cells are separated from the exhausted medium for example by tangential filtration or centrifugation and resuspended in the same original volume of fresh medium, where alkanoic acids or their salts or derivatives thereof have been added at final concentrations comprised between 0.1 and 20 mM. Alkanoic acids or their salts or derivatives thereof are added at the moment of the cell inoculum or after that. The addition of alkanoic acids or their salts or derivatives thereof, may be optionally repeated during growth or fermentation. Alkanoic acids, salts and/or derivatives are preferably chosen among: butyrate or propionate, preferably their sodium-salts, tributyrin and phenyl-butyrate. Especially preferred is butyrate and its sodium salt which is added at a preferred concentration comprised between 0.5 mM and 2.5 mM, even most preferably comprised between 1 mM and 1.5 mM.

The effect of alkanoic acids on the production of the recombinant processed protein, is further increased by lowering the temperature of the cell culture below 37° C., preferably in a range of temperatures comprised between 30° C. and 36° C., even more preferably 33° C. −35° C., most preferably 34° C.±0.5° C. Glucose levels are also checked during fermentation and preferably maintained above 1 g/L. Batch fermentation during the production phase is performed preferably according to the following parameters:

Temperature: 34°±1° C., preferentially 34°±0.5° C.

pH: 7.15±0.1

pO₂:50% ±20%

According to the preferred fermentation conditions, the higher concentration of tc-uPA is obtained after 5 days in culture, after addition of the alkanoic acid, derivatives or salts thereof. The production of active product may be followed by functional or immunological assays. tc-uPA production is followed for example by a chromogenic assay, such as the Pefachrome® assay. Alternatively the production of active tc-uPA or the disappearance of sc-uPA, may be followed by SDS-PAGE in denaturing and reducing conditions, because of a different electrophoretic migration pattern: sc-uPA migrates in fact as a ˜50-54 kD single chain polypeptide, while tc-uPA HMW is separated into the A- and B-chain, respectively migrating at ˜20 and ˜33 kD.

According to a preferred embodiment, the exhausted (i.e. the culture medium where cells have been grown) cell culture supernatant containing tc-uPA is recovered usually after 3-8 days in culture, usually at the fifth day when the balance between recombinant protein levels and cell viability (the latter kept preferably higher than 70%) is optimal. Alternatively the exhausted supernatant is recovered when sc-uPA is absent as measured by SDS reducing PAGE, and maximally converted into tc-uPA, where for tc-uPA is intended a mixture of the HMW and LMW tc-uPA. Usually the optimal time for the recovery of mature tc-uPA is comprised between 48 and 200 hours, with a preferred time of 120 hours of culture in the presence of alkanoic acids or their derivatives or salts thereof and usually corresponds to a tc-uPA production level of about 4000 IU/ml. According to the described embodiments of the invention, conversion of the precursor forms (pre-prouPA, pro-uPA, scuPA) to the catalytically active tc-uPA is characterized by an efficiency higher than 95%, as determined by analytical reducing SDS-PAGE. Of the total tc-uPA produced, about 80% is in the HMW form and the remaining 20% is in the LMW form. Therefore, further object of the present invention is a tc-uPA containing cell culture supernatant obtainable according to the process described and characterized by steps a) through c).

In one of its further embodiments the invention is related to a method for the production of recombinant catalytically active tc-uPA HMW and LMW, which results from the efficient conversion of the catalytically inactive sc-uPA or pro-uPA or Prourokinase, directly performed into the exhausted culture medium and characterized by a conversion rate of the precursor into the mature protein higher than 95%.

A further object of the present invention is represented by a chromatographic process for the isolation of recombinant tc-uPA HMW and tc-uPA LMW molecular forms characterized by the use of the cell culture supernatant obtained in step c) of the tc-uPA production process.

According to a preferred embodiment of the invention, LMW tc-uPA is separated from HMW tc-uPA by a process comprising a ion-exchange chromatography and preferably accordingly to the following additional steps: d) acidification of the cell culture supernatant with a weak acid to a pH comprised between 5.0 and 5.8, with the optional addition of a non-ionic detergent and filtration, e) contacting the supernatant with a ion-exchange chromatography column at a pH comprised between 5.5 and 6.5, f) release of the LMW tc-uPA by addition of a buffer solution with a pH value comprised between 5.5 and 6.5, further comprising a monovalent ion in concentration comprised between 200 and 300 mM, such as a 250 mM NaCl in phosphate buffer; g) release of the HMW tc-uPA by addition of a buffer solution at pH values comprised between 5.5 and 6.5 further comprising monovalent ions in concentration of at least 400 mM, such as a 500 mM NaCl phosphate buffer. Intermediate washings are also performed during chromatography to get rid of all the components not specifically related with HMW and LMW tc-UPA and are carried out with buffers and/or solutions well known in the art.

According to yet another embodiment, the invention is related to the further purification of recombinant HMW and LMW tc-uPA up to the therapeutical grade, wherein the two forms released respectively in steps g) and f of the separation process are used and which further comprises an affinity chromatography on benzamidine column. This latter enables the purification procedure to get rid of eventual trace of sc-uPA if any.

Benzamidine chromatography is usually performed according to methods well known in the art. According to a preferred embodiment of the purification process, the HMW tc-uPA is purified by benzamidine chromatography further processing the eluate obtained in step g) through the following additional steps: g′) contacting the eluate containing HMW tc-uPA with a benzamidine column at pH values comprised between 6.2 and 6.8; g″) releasing the tc-uPA HMW with a buffer solution at pH values comprised between 3.8 and 4.2, further comprising: sodium acetate in concentration ranging between 50 and 150 mM, NaCl in concentration ranging from 300 to 500 mM; g′″) further optionally contacting the released tc-uPA HMW with a gel-filtration column and releasing the HMW tc-uPA with a low-salt phosphate or acetate buffer, such as a 5 mM sodium phosphate buffer, at pH values comprised between 4 and 7.

According to yet another preferred embodiment of the invention, the LMW tc-uPA is purified by benzamidine chromatography by further processing the eluate from step f) according to the following additional steps: f) contacting the eluate containing LMW tc-uPA with a benzamidine column at pH values comprised between 6 and 8; f″) releasing the tc-uPA LMW with a solution at pH values comprised between 3.8 and 4.2 further comprising sodium acetate in concentration comprised between 50 and 150 mM, NaCl in concentration comprised between 300 and 500 mM; f′″) further optionally contacting the released tc-uPA LMW with a gel-filtration column and releasing the LMW tc-uPA with a buffer solution at pH values comprised between 4 and 7, such as a 5 mM sodium phosphate or acetate buffer.

The product(s) obtainable by the combination of the production (steps a through c), separation (steps d through g) and purification processes (steps g′ through g′″ and f′ through f′″), characterized in that the supernatant obtained from the exhausted cell culture medium obtained in step c) of the production process has been used, is recombinant tc-uPA. Recombinant tc-uPA is in the HMW molecular form, as previously defined, and is obtained at a purity level higher than 90%, and/or tc-uPA in the LMW form, as previously defined, at a purity level higher than 90%, as determined by analytical electrophoresis on SDS-PAGE.

Purified recombinant tc-uPA (HMW and/or LMW) is in the active form, as confirmed by functional and biochemical assays and has a therapeutical grade purity in compliance with the European Pharmacopoeia. Therefore it does not require any further processing and/or purification, as opposed to recombinant pro-uPA or sc-uPA produced by the recombinant DNA technologies belonging to the prior art. Its molecular form is confirmed by structural data obtained by mass spectroscopy and N-terminal analysis by Edman degradation.

As determined by functional assays such as the clot lysis assay, Michaelis-Menten constant determination, plasminogen activator inhibitor I (PAI-1) binding assay, the recombinant products produced accordingly to the present invention are functionally undistinguishable from the extractive tc-uPA as their activities in the assays are fully comparable with those of the extractive, natural product.

Moreover, they are advantageously derived from CHO cells which have a well proven safety in terms of recombinant protein production.

Purified recombinant tc-uPA HMW and LMW according to the present invention are used as powerful fibrinolytic agents for the treatment of thrombosis and for any other kind of pathological events, where it is necessary to pharmacologically remove a plasma clot. Their use is supported by the well proven clinical use of the corresponding natural extractive forms.

Therefore in a further embodiment the invention is related to the recombinant HMW/LMW tc-uPA obtainable according to the processes of the invention for the treatment of thromboembolytic events requiring the pharmacological removal of clots, such as peripheral arterial occlusion, catheter clearance, pulmonary embolism, deep venous thrombosis or for the treatment of myocardial infarction. The present invention is described in its best mode of realization by the following experimental examples.

### DESCRIPTION OF THE DRAWINGS

FIG. 1: reducing SDS-PAGE of recombinant tc-uPA. The reducing SDS-PAGE of purified tc-uPA as obtained after addition of 1.2 mM butyrate to the cell culture medium and 5 days of fermentation is shown. Under reducing conditions the tc-uPA is split in A-chain (20 KD) and B-chain (33 KD), and the sc-uPA runs at about 55 KD. Lane 1: purified Urokinase from a recombinant CHO culture grown in the absence of Na-butyrate; lane 2: purified Urokinase from a recombinant CHO culture grown for five days in the presence of 1.2 mM Na-butyrate. Growth conditions are described in the text; lane 3: Molecular weight Standard.

FIG. 2: Mass Analysis of recombinant and extractive tc-uPA. The figure shows the spectra obtained with mass spectroscopy analysis of recombinant and extractive tc-uPA (HMW and LMW) in both native (glycosylated; the two upper spectra) and deglycosylated forms (the two lower spectra).

FIG. 3: Clot lysis assay. The experiment was performed by addition of 0.5 ml human Plasma to 100 μl Urokinase (1000 U/ml) and incubated at 42° C. for 5 min. 100 μl Thrombin (20 U/ml) were added to the mixture and the absorbance was measured at 660 nm during 20 min. at 42° C.

The shaded areas from left to right at “time 0 sec” and at “time 425 sec” do represent: A) recombinant Urokinase; B) extractive Urokinase; C) plasma without addition of thrombin (no clot formation control); D) plasma with thrombin, but without addition of Urokinase (positive clot formation control).

At time 425 sec complete lysis is observed when recombinant or extractive Urokinase are present in the clot.

It is shown that recombinant and extractive Urokinase (HMW tc-uPA) exhibit the same clot lysis time.

Experimental Part

### EXAMPLE 1

### Cloning and Selection of Stable Clones Expressing Pre-Pro-UK.

The cDNA sequence encoding for the human Pre-prourokinase (corresponding to sequence ID D00244 in Genebank) was synthetized from the mRNA of a human kidney cell line (CAKl-1) according to methods well known in the art, described for example in Molecular Cloning: A laboratory Manual, Sambrook et al., Cold Spring Harbor Laboratory press, (1989).

Briefly, cDNA was synthesized by AMV Reverse Transcriptase (Böhringer-Mannheim) reaction in the presence of the mRNA mixture, Oligo dT18 (Böebehringer-Mannheim) and a mixture of the four deoxynucleotides (dATP, dGTP, dCTP, dTTP), according to the manufacturer's instructions. The mixture of cDNA molecules was specifically amplified by PCR with the following 5′ and 3′ primers:

| | |
|---|---|
| Oligo1 (5′): | ^{5′}TAGCGCCGGTACCTCGCCACCATGAGA^{3′} |
| Oligo2 (3′): | ^{5′}TGGAGATGACTCTAGAGCAAAATGACAACCA^{3′} |

The 1296 nucleotides long cDNA sequence encoding the human pre-proUK sequence was obtained and cloned into a pBR322 derived integrative expression vector carrying the following features:

the human pre-proUK cDNA sequence, under the control of the viral SV40 early promoter (Benoist C. & Chambon P. 1981, Nature 290:304-310)).

the TrpB selection marker for the metabolic selection in CHO-Messi cells (Hartman, S C Mulligan, R C (1988). Proc. Natl. Acad. Sci. USA, 85: 8047-51),

Ampicillin resistance (bla gene) as an antibiotic resistance marker in E. coli.

the E. coli origin of replication for amplification in E. coli.

The final recombinant expression vector obtained was called pTZA9.

The CHO cell line used for the production of recombinant tc-uPA was the CHO-Messi cell line (ECACC, Porton Down, Salisbury, United Kingdom, reg. n° 93080520).

This cell line is able to grow in suspension in chemically defined media CHOMaster® (Ferruccio Messi Cell Culture Technologies, Zürich, Switzerland) without addition of any serum or proteinaceous component. The duplication time of this cell line in such media (complete and selection media) is of about 24 hours. Transfection of CHO-Messi cells with pTZA9 was performed according to the method described in Felgner et al., (1987). Proc. Natl. Acad. Sci. USA, 84: 7413-7417, and respected the recommendations of the transfection's reagent manufacturer. Briefly 1 μg DNA expression vector pTZA9 (of a solution of 100 μg/ml) was mixed with 30 μl Lipofectin® (GIBCO BRL, Life Technologies) in about 500 μl exponentially growing CHO-Messi cells (about 1×10⁶ cells). The transfection mixture was kept 30 min at room temperature, before adding CHOMaster® medium supplemented with 25.1 mg/l Tryptophan and incubating overnight at 37° C.

The CHO-Messi cell line is auxotroph for Tryptophan and therefore it absolutely needs this aminoacid for growing. After the transfection of the CHO-Messi cells with the Pre-prourokinase expression vector, carrying also the Tryptophan Synthase gene, the CHO-Messi cells take advantage of their acquired ability to produce Tryptophan and do manage now to grow in a chemically defined medium in the absence of Tryptophan upon addition of serine and indol.

Stable transfected cells were obtained after limiting dilution in selective CHOMaster® medium (without Tryptophan), with the addition of serine (0.02 g/l) and indole (0.35 g/l).

Selection of the Urokinase producing clones occurred by dilution of the transfection mixture with selective CHOMaster® medium in microtiter wells. Another system used for obtaining selected single clones producing Urokinase was to dilute 1:10 a cell suspension of about 10³ cells/ml with a highly viscous solution of 0.2 g/l Methocel in selective CHOMaster® medium with addition of 4% dialysed foetal calf serum. The viscous mixture was seeded onto a 24-well cluster plate and after 2 weeks single clones were picked up with a sterile pipette tip and a new culture in suspension was started.

The following culture strategy was adopted for the preparation of the inoculum in the bioreactor. A cell culture raised in microtiter wells was further split 1:3 with selective CHOMaster® medium in larger wells (24-well cluster and 6-well cluster plates) and subsequently in 25- and 75 cm²-T flasks once the cell density reached about 4-5×10⁵ cells/ml.

Keeping constant the splitting ratio (1:3) and the cell density at which the split in the T-flasks occurred, the cultures in suspension were transferred into 2 L spinner flasks (Integra Bioscience, Switzerland) and a working volume of 1 L was used during this growth process.

### EXAMPLE 2

### Determination of the Optimal Conditions for tc-uPA Secretion/Processing.

At first, the optimal conditions related to butyrate concentration and temperature in a laboratory scale were determined. Preliminary experiments aimed at optimizing the Na-butyrate concentration were performed in a 1 L culture of the recombinant CHO cell line (1×10⁶ cells/ml), in order to achieve the best tc-uPA (HMW and LMW) production and the higher cell viability after 5 days of cultivation in spinner-flasks.

In table 1 are reported the cell viability and the uPA yield after 5 days cultivation in 1 L Spinner-flask culture at 37° C. and at different Na-butyrate concentrations. uPA activity was followed by a chromogenic assay using the Pefachrome® UK (54-46) (Pentapharm, LTD, Basel, Switzerland) as chromogenic substrate, according to the manufacturer's instructions; the cell viability was followed every day and measured microscopically after Trypan blue dye-exclusion method, as described in Doyle et al. A, Griffiths, J B and Newell, D G (Eds.) (1994), in “Cell & Tissue Culture: Laboratory Procedures”. John Wiley & Sons. New York.

TABLE 1

| Na-Butyrate in 1 L | Activity of | Cell |
|---|---|---|
| Spinner-flask culture | tc-uPA (IU/ml) | viability |
| 0 (control) | 320 | 92% |
| 0.6 mM | 526 | 83% |
| 1.2 mM | 959 | 76% |
| 2.5 mM | 361 | 56% |

As shown in Table1, the most effective Na-Butyrate concentration in terms of uPA yield is 1.2 mM after 5 days of cultivation in a 1 L Spinner-flasks at 37° C. At this concentration the cell viability is still reasonably high (76%), even if compared with the control (92%). A high cellular viability prevents a too extensive contamination of the supernatant with cellular debris and host related proteins and reduces the load of contaminants to be eliminated during the purification process.

A second set of experiments was performed at 1.2 mM butyrate, in a 5-days fermentation to determine the optimal temperature in a 2 L culture (1.5-2×10⁶ cells/ml) of the recombinant CHO cell line in bioreactor.

TABLE 2

| Temperature | Activity of tc-uPA (IU/ml) | |
|---|---|---|
| (1.2 mM Na-Butyrate) | (5 days cultivation) | Cell viability |
| 37° C. (control) | 1830 | 53% |
| 34° C. | 5043 | 67% |
| 32° C. | 800 | 84% |

In table 2 it is shown that 34° C. is the temperature which allows a high uPA productivity rate together with high cell viability. Interestingly, when the same experiment was performed at 32° C., an activity of 3961 U/ml was reached only after 12 days with lower cell viability (63%).

The reduction of the cultivation time is highly desirable for economic reasons as well as to maintain the integrity of the protein. It is in fact desirable to avoid a long exposure of the recombinant product to different proteolytic and glycolytic enzymes derived from lysed cells.

From the data shown in Table 2 it appears that lowering the cultivation temperature to 34° C., together with the addition of Na-Butyrate (1.2 mM) to the culture medium, determines a synergetic effect resulting in a better yield of active uPA.

From these data it can be deduced that the 1.2 mM Na-Butyrate treatment at 34° C. temperature are optimal conditions for active tc-uPA production and cell viability, in particular for a 5 days cultivation batch.

### EXAMPLE 3

### Production of Recombinant tc-uPA in CHO Cells in a 2 L Bioreactor.

The cell inoculum was performed in a 2.4 L bioreactor (Infors HT, Type Labforce, Bottmingen, Switzerland) by splitting an exponentially growing cell culture. The ratio “volume of inoculum/volume fresh medium” was chosen between 1:1 and 1:5, according to the total capacity of the bioreactor and to the culture conditions. A working volume of 2 L and a final cell density of the inoculum of 3×10⁵ cells/ml were chosen.

The culture used for the primary inoculum had a viability ratio not lower than 95%. During the growth in the bioreactor the culture in suspension was monitored for the concentration of glucose, which was never kept lower than 1 g/L in the exhausted medium.

The following fermentation parameters were set up for the culture:

| Temperature: | 37° ± 0.5° C., |
|---|---|
| pH: | 7.15 ± 0.1 |
| pO₂: | 50% ± 20% |

When the cellular density reached values of about 2×10⁶ living cells/ml, the cells were separated from the exhausted medium by tangential filtration (or centrifugation). Cells were then resuspended in the bioreactor in the same original volume of fresh medium CHOMaster® with the addition of sodium butyrate to achieve a final concentration of 1.2 mM.

The temperature of the culture was lowered to 34°±0.5° C., and the other fermentation parameters were kept as previously set, as follows:

| Temperature: | 34° C. ± 0.5° C., |
|---|---|
| pH: | 7.15 ± 0.1 |
| pO₂: | 50% ± 20% |

The production of active u-PA (tc-UPA HMW and LMW) was monitored by a chromogenic test on a specific substrate, Pefachrome® UK. A progressive increase of the activity was observed up to maximal values as high as 7000 IU/ml, achieved after 4-5 days of fermentation. At this point the cells were harvested and subsequently discarded and the exhausted culture medium, containing tc-uPA, was further processed for purification.

The average production levels relative to four independent fermentations are presented in table 3:

TABLE 3
Fermentation data

| Days after the | Activity on the | |
|---|---|---|
| addition of 1.2 mM | chromogenic substrate | |
| Na-butyrate | (IU/ml) | Cell viability |
| 1 | 155 ± 23% | 87 ± 6% |
| 2 | 662 ± 32% | 81 ± 12% |
| 3 | 1995 ± 13% | 78 ± 15% |
| 4 | 3170 ± 23% | 72 ± 14% |
| 5 | 5043 ± 13% | 67 ± 10% |

In the bioreactor a gradual decrease of the cell viability was observed from the first to the fifth day in culture after the addition of sodium butyrate. The minimal cell viability value at 1.2 mM butyrate was observed at the fifth day in culture and was not significantly lower than 70% and is therefore still relatively high. At these values a limited release of lysosomal degradation enzymes into the growth medium is expected.

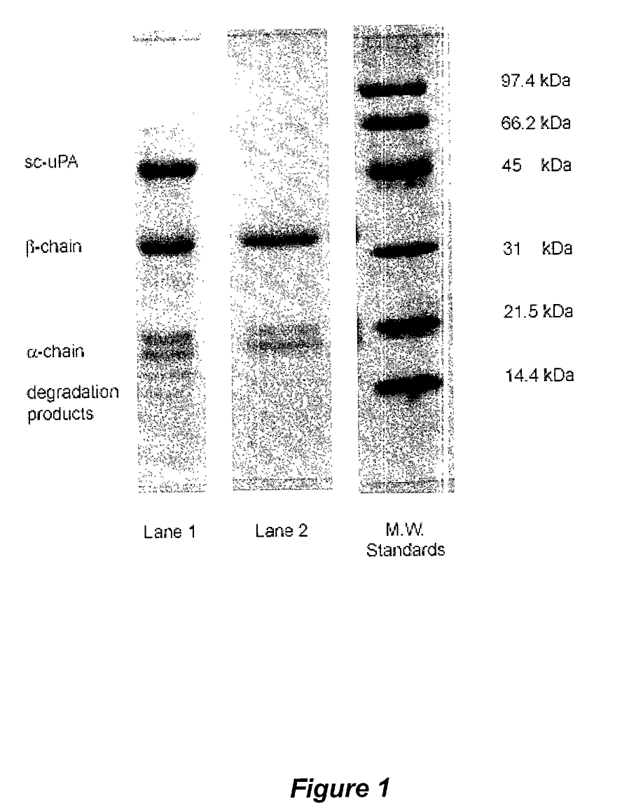

As shown in table 3, the dramatic increase in the expression of tc-uPA during the production process as it is described, occurs mainly during the last 2/3 days of fermentation after the addition of sodium butyrate. The permanence of most of the secreted Urokinase into the exhausted medium, is optimal when limited to 2/3 days, at a temperature of 34° C. This combination of parameters reduces the exposure of the protein to the degradation activity of the proteolytic and glycolytic enzymes and allows for good quality of the recombinant protein to be purified. In FIG. 1 are shown the products obtained after fermentation in the absence or in the presence of butyrate 1.2 mM, at a temperature of 34° C. In the latter conditions the complete conversion of sc-uPA into tc-uPA can be appreciated, when compared to a culture where no Na-butyrate was added.

### EXAMPLE 4

### Purification of tc-uPA HMW and LMW

Purification of tc-uPA HMW.

The supernatant of the cell culture grown in bioreactor, obtained as described in Example 3 was acidified by addition of CH₃COOH to a pH of 5.5 and cleared from cellular debris by filtration on 0.45 μm filter. Tween-80 0.01% was added and the supernatant was loaded onto an ion exchange chromatography column (SP Sepharose Big Beads, Amersham-Pharmacia) previously equilibrated with a 20 mM pH 6.0 sodium phosphate buffer solution. The column bed size was 10 cm height, 2.6 cm diameter. The flow rate during loading and wash was 10 ml/min and during elution was 2 ml/min.

After loading, the column was first washed with 3 volumes of 20 mM sodium phosphate, 150 mM NaCl, pH 6 buffer solution, in order to remove the non-Urokinase related impurities and subsequently with 3 more volumes of 20 mM sodium phosphate, 250 mM NaCl, pH 6.0 buffer solution, in order to mainly elute tc-uPA LMW. This latter is stored frozen for a further purification.

The elution of tc-uPA HMW was performed by passing through the ion exchange column, a 20 mM sodium phosphate, 500 mM sodium chloride, pH 6.0 buffer solution.

The eluate obtained, containing Urokinase (tc-UPA HMW) was brought to pH 6.5 by the addition of 1 N NaOH. Then it was loaded onto a benzamidine Sepharose 6B affinity column, previously equilibrated with at least 2 volumes of a 20 mM sodium phosphate, 400 mM NaCl, pH 6.5 buffer solution.

The bed size of the column was 10 cm height, 2.6 cm diameter. The flow rate during the load, wash and elution steps was 2.5 ml/min.

The column was subsequently washed with 2 volumes of a 20 mM sodium phosphate, 400 mM NaCl, pH 6.5 buffer solution and finally eluted with 2.5 volumes of 100 mM sodium acetate, 400 mM NaCl, pH 4.0 buffer solution.

The benzamidine column allowed to get rid of the non-Urokinase impurities, as well as of the undetectable amount, if any, of sc-uPA that could be present in the load.

The tc-uPA HMW containing fractions were identified as belonging to a relatively consistent and unique peak in the chromatogram and were pooled together. The obtained pool was loaded onto a gel filtration column (separation on the basis of molecular size exclusion). The gel filtration column had 30 cm height and 2.6 cm diameter, and was previously equilibrated with a 5 mM sodium phosphate pH 4.9 buffer solution. The applied flow rate was about 3 ml/min.

Urokinase tc-uPA HMW was thus eluted in its pure form, by running the elution with a 5 mM sodium phosphate pH 4.9 buffer solution, as shown in FIG. 1 (lane2).

Urokinase was finally ready to be formulated in a buffer suitable for a terminal lyophilisation.

Purification of LMW tc-uPA

The fraction(s) corresponding to the tc-uPA LMW, collected from the wash of the ion exchange column with 20 mM sodium phosphate, 250 mM NaCl, pH 6.0 buffer solution, were pooled and subsequently purified through an affinity benzamidine chromatography column. Before being loaded onto this column, the pool was adjusted to pH 6.5 or 7.0 by addition of 1N NaOH and the column was previously equilibrated with at least 2 volumes of a 20 mM sodium phosphate, 400 mM NaCl, pH 6.5 buffer solution. After the load, the column was washed with 2 volumes of 20 mM sodium phosphate, 400 mM NaCl, pH 6.5 buffer solution and finally eluted with 2.5 volumes of 100 mM sodium acetate, 400 mM NaCl, pH 4.0 buffer solution. The tc-uPA LMW containing fractions were identified as belonging to a relatively consistent and unique peak in the chromatogram and were pooled together.

The obtained pool is loaded onto a gel filtration column (separation on the basis of molecular size exclusion) of 30 cm height and 2.6 cm diameter, previously equilibrated with a 5 mM sodium phosphate pH 4.9 buffer solution. The applied flow rate is about 3 ml/min.

Urokinase tc-uPA LMW is thus eluted in its pure form, by running the elution with a 5 mM, sodium phosphate pH 4.9 buffer solution and it is finally ready to be formulated in a buffer suitable for a terminal lyophilisation.

### EXAMPLE 5

### Characterization of Recombinant tc-uPA.

Processing of the Recombinant Molecule

Characterization of recombinant tc-uPA HMW was carried out in comparative studies with the commercial extractive tc-uPA HMW (Ukidan®, Serono) by mass spectroscopy and functional studies.

Molecular Mass

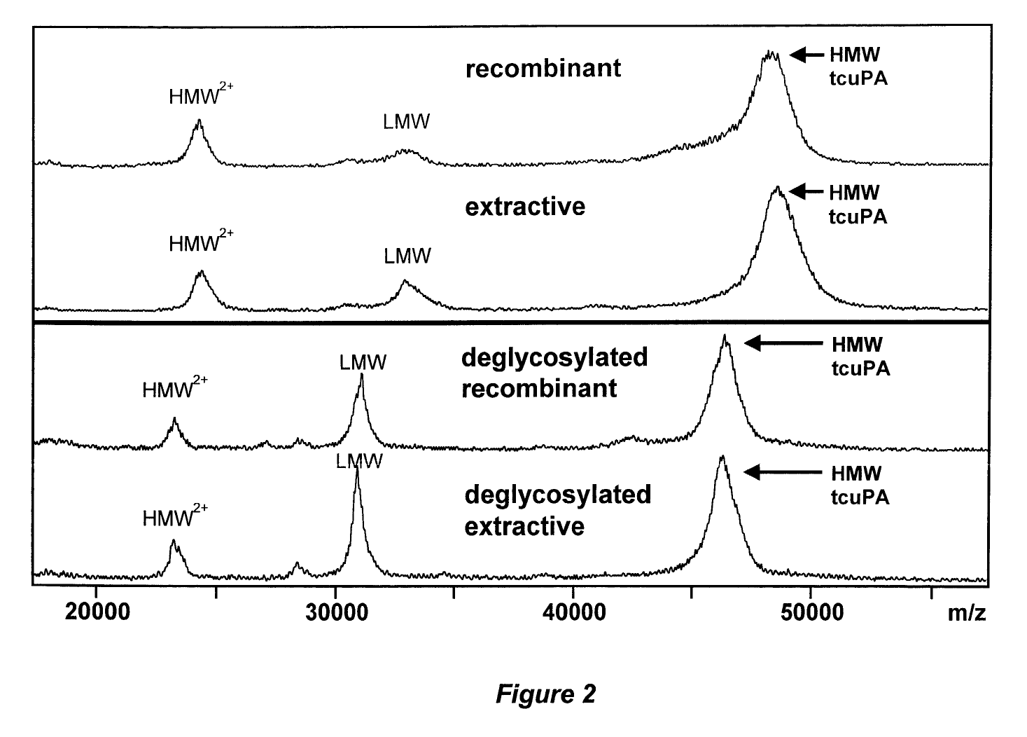

The mass spectroscopy data, the accuracy of which may vary in the range of +/−50 Da to +/−100 Da, confirmed that the molecular mass corresponded to what expected after correct processing of the precursor (sc-uPA) protein and in particular, as shown in FIG. 2, that:

the recombinant, produced accordingly to the process of the invention, and the commercial extractive HMW tc-uPA had very similar molecular masses of 48267 Da and 48565 Da, respectively;

when deglycosylated, the two forms had also very similar molecular masses of 46382 (recombinant) and 46313 Da (extractive).

similarly, the analysis of the recombinant produced accordingly to the process of the invention and extractive LMW tc-uPA exhibited very close masses in the glycosylated (33249 Da and 33189 Da, respectively) and not glycosylated form (31029 and 30969, respectively).

Moreover, MALDI-MS mass spectroscopy analysis showed that the purified molecules are largely intact (>95%), in other words it confirmed that the uPA-related degradation products are present in non-relevant amount and that therefore the chosen purification procedures do not affect the integrity of the recombinant molecule.

The correct processing at the N-terminal was confirmed through the Edman degradation of purified HMW tc-uPA. As expected, the NH₂-terminal sequence of the B-chain was determined as:

-IIGGEF-,

whereas the NH₂-terminal sequence of the A-chain was, as expected:

-SNELHQ-,

These data demonstrated that the proteolytic cleavage occurs exactly and specifically at the Lys¹⁵⁸-Ile¹⁵⁹ bond, and Lys¹⁵⁸ is correctly removed from the rest of the molecule. Moreover, the analysis of the peptide mapping confirmed the existence of correct NH₂- and C-termini of both A- and B-chains of the recombinant tc-uPA HMW

Glycosylation Pattern

Glycans were analyzed on purified recombinant tc-uPA by mass spectroscopy and fluorescence-assisted carbohydrate electrophoresis (FACE). Both methods revealed stable glycosylation: recombinant Urokinase-derived N-glycans consist of core-fucosylated, two-, three- and four antennary complex chains with a sialylation degree of 80-90%. The glycosylation site was determined by mass spectroscopy, and confirmed that the glycosylation at Asn³⁰² on tc-uPA has occurred.

Functional Studies

The biological activity of recombinant HMW tc-uPA was also determined by measuring:

analysis of K_{d} of binding to natural Urokinase receptor (see Table 4);

analysis of the stoichiometric inhibitory activity of Plasminogen activation inhibitor (PAI-1), which is the natural inhibitor of Urokinase;

analysis of the kinetic of inhibition by PAI-1 (see Table 4);

study of enzymatic parameter Kₘ on chromogenic substrate (see Table 4);

study of plasminogen activation kinetics;

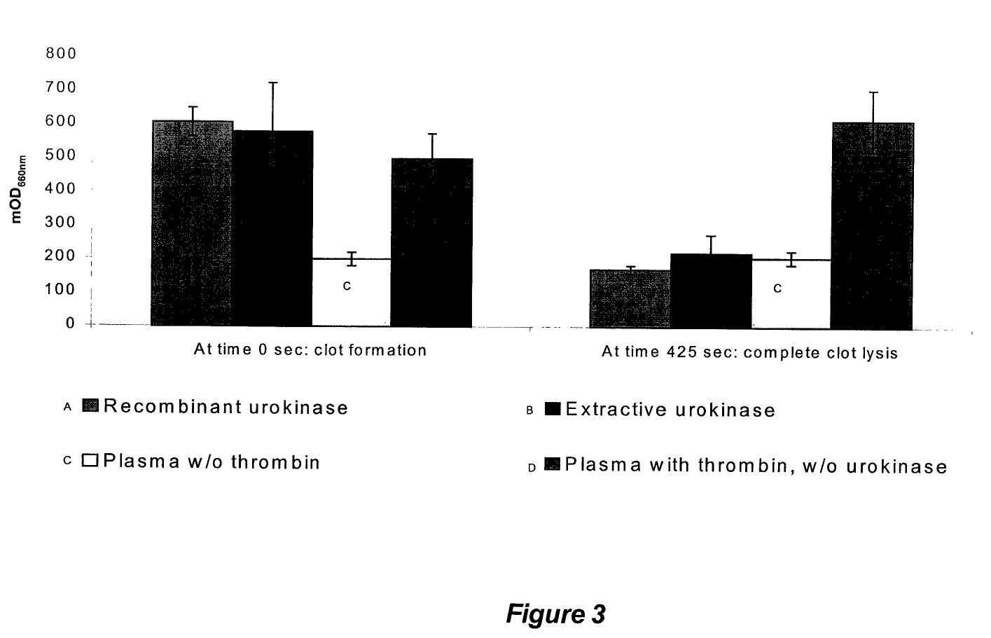

clot lysis capacity (FIG. 3);

fibrin degradation.

TABLE 4

| Sample | k₁ (M⁻¹*s⁻¹)*10⁻⁷ | Kₘ (mM) | K_{d} (nM) |
|---|---|---|---|
| recombinant HMW tc-uPA | 2.07 ± 0.57 | 0.027 ± 0.0018 | 1.24 |
| Extractive HMW tc-uPA | 2.59 ± 0.20 | 0.028 ± 0.0050 | 1.77 |

The data shown in table 4 demonstrate the substantial functional identity of the recombinant HMW tc-uPA, produced accordingly to the process of the invention, and the commercial extractive HMW tc-uPA. In particular it is shown that the two products had:

(a) similar rate constants (k₁) for the complex formation with PAI-1, as measured according to Chmielweska et al. Biochem. J. 1988, 251:327-332,

(b) similar Michaelis-Menten constants (Kₘ) for the Urokinase substrate, as measured according to Briggs, GE and Haldane JBS, Biochem. J. 1925, 29:338-339 and Lijnen, HR et al. Eur. J. Biochem. 1994, 224:567-574, and

(c) similar affinity constants for the Urokinase receptor (K_{d}), as measured according to: Cubellis, M. V. et al., J. Biol. Chem., 1986, 261:15819-15822.

1. A method for the production of a mature recombinant protein into the culture medium of an eukaryotic cell line genetically transfected with a cloned precursor cDNA sequence, comprising an incubation of said cell line in the cell culture medium wherein alkanoic acids, their derivatives or salts thereof have been added for a time of at least 24 hours.

2. A method according to claim 1 wherein said cDNA sequence encodes for a protein precursor.

3. A method according to claim 2 wherein said precursor cDNA sequence encodes for the human Pre-prourokinase.

4. A method according to claim 1 wherein said mature recombinant protein is two chain-uPA (tc-uPA).

5. A method according to claim 4 wherein the two chain uPA is HMW.

6. A method according to claim 4 wherein the two chain uPA is LMW.

7. A method according to claim 4 wherein said alkanoic acids and/or their salts and/or derivatives thereof are chosen among: butyric acid, sodium butyrate, sodium propionate, magnesium butyrate, tributyrin and phenyl-butyrate.

8. A method according to claim 7 wherein said eukaryotic cell line is a mammalian cell line chosen among: HEK-293, CV-1, COS, BSC-1, MDCK, A-431, CHO, BHK, CHO-Messi.

9. A method according to claim 8 wherein said time is comprised between 48 and 200 hours.

10. A method according to claim 8 wherein said cell culture is serum-free.

11. A method according to claim 8 wherein said incubation is performed at a temperature equal or lower than 37° C.

12. A process for the production of recombinant tc-uPA comprising the following steps:
a) culturing genetically manipulated CHO cells stably transfected with the Pre-prourokinase cDNA in a culture media comprising alkanoic acids or their derivatives or salts thereof, at a temperature comprised between 30° C. and 37° C.;
b) continuing said cell-culture for a period of time of at least 24 hours;
c) recovering the cell culture supernatant.

13. A process according to claim 12 wherein said period of time in step b) is comprised between 72 and 150 hours.

14. A process according to claim 12 wherein cell viability of said CHO cell-culture in step b) is at least 70%.

15. A process according to claim 12 wherein said temperature is comprised between 33° C. and 35° C.

16. A process according to claim 12 wherein said alkanoic acid derivative is chosen among: butyric acid, sodium butyrate, sodium propionate, magnesium butyrate, tributyrin, phenyl butyrate, at concentration comprised between 0.1 mM and 20 mM.

17. A process according to claim 16 wherein said CHO cells are CHO-Messi cells.

18. A process according to claim 17 wherein in step a) said culture media is a serum free culture medium.

19. A process for the isolation of recombinant HMW and/or LMW tc-uPA from an exhausted culture media of genetically engineered CHO cells characterized by using the cell culture supernatant obtained according to claim 17.

20. A process according to claim 19 wherein said isolation comprises a ion-exchange chromatography.

21. A process according to claim 20 for the separation of recombinant HMW from LMW tc-uPA further comprising the steps of:
d) acidification of the cell culture supernatatant with a weak acid to pH values comprised between 5 and 5.8, optionally adding a non-ionic detergent;
e) contacting the acidified supernatant with a ion-exchange chromatography column at pH values comprised between 5.5 and 6.5;
f) releasing the LMW tc-uPA by addition of a buffer solution with a pH value comprised between 5.5 and 6.5, comprising a monovalent ion in concentration comprised between 200 and 300 mM;
g) releasing the HMW tc-uPA by addition of a buffer solution with a pH value comprised between 6-7.5, comprising monovalent ions in concentration of at least 400 mM.

22. A process according to claim 21 wherein the acidified supernatant in step d) is additionally filtered.

23. A process according to claim 21 wherein said isolation further comprises a benzamidine chromatography.

24. A process according to claim 23 for the purification of recombinant tc-uPA HMW comprising the steps of:
g′) contacting the released HMW tc-uPA containing buffer solution in step g) with a benzamidine column, at pH values comprised between 6.2 and 6.8
g″) releasing the tc-uPA HMW with a buffer solution with a pH value comprised between 3.8 and 4.2, further comprising monovalent ions in concentration comprised between 300 and 500 mM;
g′″) further optionally contacting the released tc-uPA HMW with a gel-filtration column and releasing of the HMW tc-uPA with a low-salt solution buffer at pH values comprised between 4 and 7.

25. A process according to claim 23 for the purification of recombinant tc-uPA LMW further comprising the additional steps of:
f′) contacting the released LMW tc-uPA containing solution obtained in step f), with a benzamidine column, at pH values comprised between 6 and 8;
f″) releasing the tc-uPA LMW with a buffer solution with pH values comprised between 3.8 and 4.2 further comprising monovalent ions in concentration comprised between 300 mM and 500 mM;
f′″) further optionally contacting the released tc-uPA LMW with a gel-filtration column and releasing the LMW tc-uPA with a low-salt solution buffer at a pH comprised between 4 and 7.

26. Recombinant tc-uPA obtainable by the process according to claim 12.

27. Recombinant tc-uPA obtainable by the process according to claim 18.

28. Recombinant HMW and LMW tc-uPA product obtainable by the process according to claim 21.

29. Recombinant HMW and LMW tc-uPA product obtainable by the process according to claim 23.

30. Recombinant purified HMW tc-uPA obtainable by the process according to claim 24.

31. Recombinant purified LMW tc-uPA obtainable by the process according to claim 25.

32. A method for the treatment of thromboembolytic disorders wherein recombinant HMW tc-uPA according to claim 30 is used.

33. A method for the treatment of thromboembolytic disorders wherein recombinant LMW tc-uPA according to claim 31 is used.

34. A method according to claim 32 wherein said disorders are chosen among: peripheral arterial occlusion (PAOD), catheter clearance, pulmonary embolism, deep venous thrombosis.

35. A method according to claim 33 wherein said disorders are chosen among: peripheral arterial occlusion (PAOD), catheter clearance, pulmonary embolism, deep venous thrombosis.

36. A method for the treatment of myocardial infarction wherein HMW tc-uPA according to claim 30 is used.

37. A method for the treatment of myocardial infarction wherein LMW tc-uPA according to claim 31 is used.

38. Pharmaceutical compositions comprising as an active agent the recombinant HMW tc-uPA according to claim 30.

39. Pharmaceutical compositions comprising as an active agent the recombinant LMW tc-uPA according to claim 31.


SEQUENCE LISTING (2 sequences)

SEQ ID NO: 1 - DNA, 27 bp, Artificial Sequence
  Description of Artificial Sequence human preprourokinase cDNA PCR amplification primer 5′ end
tagcgccggtacctcgccaccatgaga

SEQ ID NO: 2 - DNA, 31 bp, Artificial Sequence
  Description of Artificial Sequence human preprourokinase cDNA PCR amplification primer 3′ end.
tggagatgactctagagcaaaatgacaacca